(19) United States
(12) Patent Application Publication
(10) Pub. No.: US 2018/0035925 A1
(43) Pub. Date: Feb. 8, 2018

(54) Method For Hearing Performance Assessment and Hearing System

(71) Applicant: Sonova AG, Staeffa (CH)

(72) Inventor: Michael BORETZKI, Rüti (CH)

(21) Appl. No.: 15/552,314

(22) PCT Filed: Mar. 3, 2015
(86) PCT No.: PCT/EP2015/054443

§ 371 Date: Aug. 21, 2017

(51) Int. Cl. A61B 5/12; H04R 25/00

(52) U.S. Cl. CPC A61B 5/123; H04R 25/70; H04R 25/356; H04R 25/353; H04R 25/552; H04R 2225/43; H04R 2225/61

(57) ABSTRACT

Provided is a method for hearing performance assessment of a user wearing a hearing system (10) comprising at least one hearing aid. Said method comprises the steps of picking up (12; 12′, 12″) sound from the environment thus providing a signal, processing (16; 16′, 16″) the signal thus obtaining a processed signal, and presenting (14; 14′, 14″) the processed signal to the user by speaker of the hearing aid. Said processing comprises applying at least one type of modulation to the signal.

### TECHNICAL FIELD

The present invention is related to a method for hearing performance assessment of a user wearing a hearing system.

### BACKGROUND OF THE INVENTION

Hearing devices are typically small ear-level devices used to improve the hearing capability of hearing impaired people. This is achieved by picking up the surrounding sound with a microphone of a hearing device, processing the microphone signal thereby taking into account the hearing impairment of the user of the hearing device and providing the processed sound signal into the ear canal of the user via a miniature loudspeaker, commonly referred to as a receiver.

It should be noted that in the context of hearing aids a signal can be an acoustical sound or a digital representation of sound. A signal is characterized by amplitude and frequency.

In order to improve said signal processing, hearing performance assessment is important in developing improved hearing solutions and in validating aided hearing with hearing impaired users. Typically, hearing performance of a user is assessed in the office of a hearing care professional by using assessment methods. Commonly used methods comprise audibility measurement with pure tones, loudness discomfort measurement with pure tones, speech intelligibility measurements in quiet, speech intelligibility measurements in noises, which have been designed for maximum reliability of measurement.

Commonly known assessment methods are performed by using specific equipment, for example sound generators, sound data storages, presentation functionality, loudspeakers and headphones. Assessment methods can be performed using the hearing aid as sound generator and presentation functionality. Hearing aids may create unwanted artifacts, for example a “vibrato” to sounds with steady-state pitch being created, wherein the mixture of direct sound via a vent and processed sound being shifted by a reduced frequency range (by a small number of Hz) for suppressing feedback probably perceived by the user.

Application of amplitude modulation to acoustic signals is known in audio signal processing. Also known is application of left-right balance modulation, as well as variation of center frequencies of formants of voiced speech signals. Examples of modulated sounds are in the so called warble tones which are sinusoids with modulated frequency. These warble tones are used in case of measurements are performed by using loudspeakers and narrowband stimuli shall be applied, wherein standing waves in the room shall not bias the measurement results. From psycho-acoustical research it is known to measure amplitude or frequency modulation thresholds with technical sounds, for example white noise, pink noise, narrowband noise and pure tunes.

As mentioned above, commonly known hearing performance assessments are performed in the hearing care professional office. However, due to laboratory like conditions or rather environments in the hearing care professional office, hearing performance assessment suffers to capture real life hearing. In other words, hearing care professional office environments do not cover all relevant fields of everyday life hearing. Such relevant fields of everyday life hearing comprise audibility, distinguishability, localizability, recognizability, familiarity and loudness comfort of relevant sounds.

It is therefore an object of the present invention to provide a method for hearing performance assessment of a user and a hearing system for performing said method omitting the disadvantages in the prior art.

### SUMMARY OF THE INVENTION

The following provides a definition of respective wording used in the present specification:
Spectral audibility of a signal by a user is measured by successively applying a short (e.g. 1s) modulation of the amplitude of the signal within a particular frequency band with a particular modulation frequency and modulation amplitudes varied according to a staircase procedure. The user is prompted to listen to the modulated signal and to indicate if the modulation is audible. As the amplitudes of everyday life soundscapes vary, the result of this measurement is audible modulation amplitudes across sound levels. A more detailed measurement also includes lower frequency spectral levels as factor of the modulation thresholds.
Spectral distinguishability is measured the same way as spectral audibility except that instead of amplitude modulation spectral peak modulation is applied with its center frequency being varied.
Spatial distinguishability is assessed the same as spectral audibility assessment except that instead of the amplitude modulation a binaural phase modulation is applied across the whole frequency range or at least up to 900 Hz.
Spectral spatial distinguishability is assessed the same way as spatial distinguishability except that instead of broadband phase modulation, the phase is only modulated in one frequency band.
Spatial immission direction recognizability is assessed by adding narrowband stimuli with binaural level and phase differences as they occur with different immission angles. The user indicates with a portable device from which he feels the additional stimulus is coming from. This requires technical spatial localization of this device relative to the spatial position of the hearing aids.
Sound recognizability is assessed by adding sounds of well-known or familiarized kinds of sound sources, e.g. dog barking, car hooting, nonsense syllables, etc. The user is indicating what he feels he has heard.

The present invention is directed to a method for hearing performance assessment of a user wearing a hearing system comprising at least one hearing aid. The method comprises: picking up sound from the environment thus providing a signal, processing the signal thus obtaining a processed signal, and presenting the processed signal to the user by a speaker of the hearing aid, wherein said processing comprises applying at least one type of modulation to the signal.

Hearing aids are typically used to compensate for hearing loss of a user. The invention is mainly intended to be used with hearing impaired users, it is however also possible to use the invention for a hearing performance assessment of normal hearing users.

The inventive method can be executed with examples of relevant sounds taken from real life or directly in real life. A signal is generated by picking up sound with the microphones of a hearing aid and includes digitalization of the captured sound. The signal is a digital representation of an acoustical signal. The inventive method allows to assess audibility, distinguishability, localizability, recognizability, familiarity and loudness comfort of sounds. An input means can be provided for allowing the user to indicate whether he has heard the modulation or not. Said input means can be part of the hearing system or can be provided remotely. In an example of the method, the user can be prompted to get his attention relating to the detection task before one of said at least one type of modulation will be applied.

While the invention may be carried out by using a hearing system comprising only a hearing aid it should be pointed out, that a hearing system may comprise auxiliary devices which may be used for example as interface. The hearing system comprises a processor and a memory containing program code in order to perform the invention. For the nature of the invention it is not essential, in which part of the hardware the signal processing occurs, it is however important, that the sound, which is presented to the user is delivered from a speaker of the hearing aid. The hearing system may comprise hearing aids for the left and right ear of the user.

Summarized, provided is a hearing performance assessment in real life by using the hearing system comprising the at least one hearing aid. Such method has the advantage in that all relevant fields of everyday life hearing are covered. Real life testing involves a large advantage over laboratory testing, since the sounds, involving the material for discrimination testing, are the ones which the hearing impaired user lives with. Therefore, compared to the prior art, there are remarkably less validity questions to be answered. Therefore, everyday life hearing performance can be checked easily in everyday's situation.

In an embodiment of the proposed method, said at least one type of modulation comprises: modulation of an amplitude of the signal, modulation of center frequencies of one or more spectral peaks of the signal, and/or modulation of a phase difference and/or level difference of two hearing aids. In an example, amplitude modulation and center frequency modulation are used in assessing spectral audibility and spectral discriminability of sounds.

In an embodiment of the proposed method, said applying step comprises applying said at least one type of modulation to different frequency ranges of the signal to be processed by the at least one hearing aid.

In an embodiment of the proposed method, said different frequency ranges of the signal comprise: particular frequency ranges of said signal, particular input level ranges in the time course of said signal, and particular ranges of possible modulation spectra in the time course of said signal. Hence, amplitude modulation can be applied to different frequency ranges of the signal. The number of different frequency ranges, the bandwidth as well as the band gap between subsequent frequency ranges can be selected appropriately. As an alternative, amplitude modulation can be applied to the complete bandwidth of the hearing aid.

In an embodiment of the proposed method, the hearing system comprises a left and a right hearing aid, and said applying step comprises applying said at least one type of modulation to said left and right hearing aids separately or to both thereof equally or weighted.

In an embodiment of the proposed method, the step of processing the signal further comprises modifying the signal in order to obtain a hearing loss compensation of the user, and wherein said modifying the signal is carried out prior to the step of applying or afterwards. Hence, locations in the signal path in which amplitude, center frequency and binaural modulation can be applied can be selected before hearing loss compensation processing or afterwards, i.e. subsequent to the modulation of the processed signal.

In an embodiment of the proposed method, said applying step further comprises applying the at least one type of modulation to a portion of the signal with stepwise varied parameters of the modulation for determining a modulation detection threshold for the user and the modulated portion of the signal.

In further embodiments, the proposed method further comprises the step of monitoring, by means of the input user information, spectral input levels and modulation spectra of the acoustic properties of the input signal. Said monitoring can be performed by the user or fitter by inputting whether the modulation is heard or not. An input means can be provided allowing the user or fitter to perform said input. The modulation detection threshold can be measured for selecting proper times for executing the modulation detection assessment. In a further example, the modulation detection threshold can be measured for storing trial results in addition to said signal data. Said trial results can be defined if the modulation is detected or not. In an example, the method can analyze results of the modulation detection trials such to allow interpolation of the modulation thresholds for a particular spectral input level. In a further example, the method can select a particular modulation already existing in the acoustic property which has been selected for modulation detection threshold testing.

Moreover, the present invention is directed to a hearing system exhibiting the advantages of the proposed method for hearing performance assessment of a user wearing the hearing system. According to the present invention, the hearing system comprises at least one hearing aid, the hearing system comprising a memory which contains program code for performing the method of claim 1. Hence, a hearing system is provided allowing execution of hearing performance assessment of a user by providing hearing performance assessment in real life. The hearing system can provide functionality for executing several assessment methods comprising spectral audibility, spectral distinguishability, spatial distinguishability, spectral, spatial distinguishability, spatial immission direction recognizability and/or sound recognizability.

It is expressly pointed out that any combination of the above mentioned embodiments is subject of further possible embodiments. Only those embodiments are excluded that would result in a contradiction.

### BRIEF DESCRIPTION OF THE DRAWINGS

The present invention is further described with reference to the accompanying drawings jointly illustrating various exemplary embodiments which are to be considered in connection with the following detailed description. What is shown in the figures is the following:

FIG. 1 is a schematic diagram illustrating the relationship between spectral levels and frequency in the case of applying amplitude modulation and center frequency modulation, each modulation applied to distinct frequency ranges of the input signal;

FIG. 2 is a schematic diagram illustrating the relationship between spectral levels and frequency in the case of applying amplitude modulations, each modulation applied to two frequency ranges of the input signal separated from each other;

FIG. 3 is a schematic diagram illustrating modulation detection threshold values recorded at a plurality of spectral levels;

FIG. 4 is a schematic diagram illustrating spatial modulation, and

FIGS. 5A,B are schematic diagrams representing binaural and monaural hearing systems, respectively.

### DETAILED DESCRIPTION OF THE INVENTION

FIG. 1 is a schematic diagram illustrating the relationship of spectral levels versus frequency in the case of applying amplitude modulation and center frequency modulation, each modulation applied to distinct frequency ranges of the input signal separated from each other. In a frequency range indicated by reference sign 1, amplitude modulation of the input signal is performed. In a different frequency range indicated by reference sign 2, center frequency modulation of the input signal is performed. Both modulations refer to spectral audibility and spectral discriminability of sounds. Spectral audibility (refer to frequency range 1) is measured by successively applying a short amplitude modulation during a short time interval, for example one second. The user is prompted to listen to this modulation and to indicate whether this modulation is audible or not. The spectral distinguishability (refer to frequency range 2) is measured the same way as spectral audibility, however, instead of amplitude modulation, spectral peak modulation is applied with its center frequency being varied.

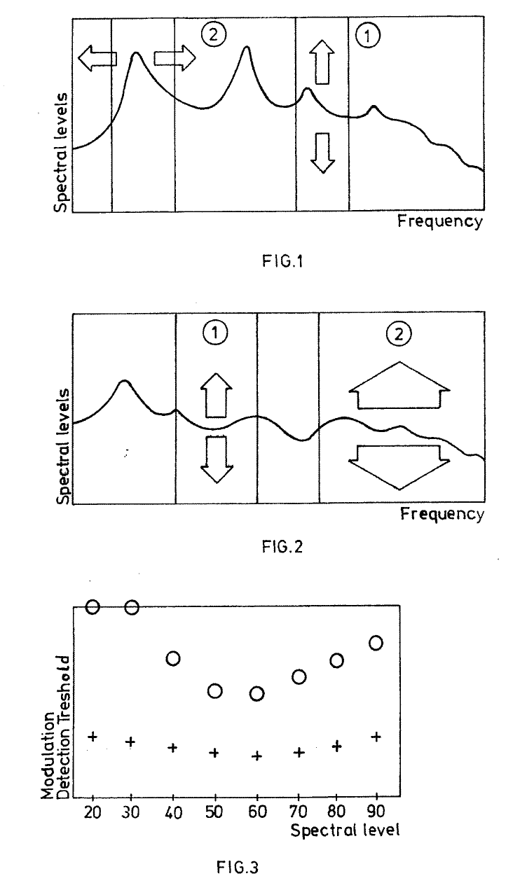

FIG. 2 is a schematic diagram illustrating the relationship of spectral levels versus frequency in the case of applying amplitude modulations, each modulation applied to distinct frequency ranges of the input signal. Contrary to the schematic diagram shown in FIG. 1, solely amplitude modulations of spectral components (refer to frequency ranges 1 and 2) of the input signal are performed, which frequency ranges are spaced apart from each other and may be different in bandwidth. In particular, frequency range 1 is smaller in bandwidth than frequency range 2. While not shown, amplitude modulation can also be applied to the complete frequency bandwidth of the hearing aid.

FIG. 3 is a schematic diagram illustrating plots showing values of modulation detection thresholds recorded at different spectral levels. In this diagram, the plot of hearing system user data is indicated by cycles, wherein the plot of normal hearing reference data is indicated by crosses. The modulation detection thresholds depend on input levels. According to the invention, modulation detection thresholds of the user wearing the hearing system are each measured based on several input levels or input level ranges. The normal hearing reference data is used for calculating real life disability in modulation detection.

The modulation detection thresholds, which are part of the described hearing performance testing assessment principle, depend on acoustic properties of the signal being processed by the hearing aid. Said acoustic properties of the signal comprise spectral levels and modulation spectrum of the spectral bands of the signal, especially of that part which is selected for the modulation detection assessment.

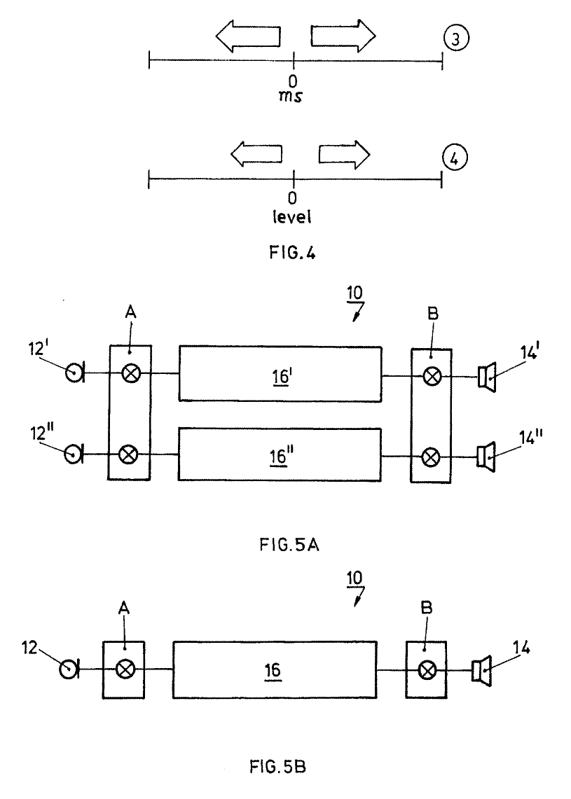

FIG. 4 is a schematic diagram showing spatial modulation. In this diagram, the upper part of the figure, indicated by reference sign 3, shows modulation of relative delay of left and right hearing aids. Further, the lower part of the figure, indicated by reference sign 4, shows modulation of relative level of the left and right hearing aids. The modulation types both refer to discriminability of spatial hearing. According to an aspect of the invention, the hearing system is capable to apply both types of modulation, wherein the parameters thereof are set differently. Parameters of the modulation can comprise depths of the modulation and the frequency or period of the modulation.

FIG. 5A shows a schematic configuration of the signal processing path of a binaural hearing system 10 comprising two hearing aids, illustrating the signal path between microphones and receivers. A sound to signal conversion step 12′, 12″ is performed by microphones. The signal processing within the hearing system 10 comprises a hearing loss compensation step 16′, 16″. Finally, a signal to sound conversion step 14′, 14″ is performed by the receivers. According to an aspect of the invention, the at least one type of modulation of the signal can be applied before the hearing loss compensation step 16′, 16″, i.e. in a location of the signal path indicated by reference sign A. As an alternative, the at least one type of modulation of the one or more acoustic properties of input signals can be applied subsequent to hearing loss compensation means 16′, 16″, i.e. in a location of the signal path indicated by a reference sign B. Hence, the inventive hearing performance testing measurement schemes can be applied to the unprocessed signal (location A) or to the processed signal (location B). The first application variant allows to determine if different processing schemes do affect discrimination abilities of the user differently. The second application variant allows to determine the users aided discrimination abilities for auditory signals.

While these variants have been described for a binaural hearing system, the concept of applying a modulation prior or after the step of hearing loss compensation can be used also within a single hearing aid or rather monaural hearing system 10 as shown in FIG. 5B. In particular, FIG. 5B shows a schematic configuration of the signal processing path of a monaural hearing system 10 comprising one hearing aid, illustrating the signal path between microphones and receivers. A sound to signal conversion step 12 is performed by microphones. The signal processing within the monaural hearing system 10 comprises a hearing loss compensation step 16. Finally, a signal to sound conversion step 14 is performed by the receivers. According to an aspect of the invention, the at least one type of modulation of the signal can be applied before the hearing loss compensation step 16, i.e. in a location of the signal path indicated by reference sign A. As an alternative, the at least one type of modulation of the one or more acoustic properties of input signals can be applied subsequent to hearing loss compensation means 16, i.e. in a location of the signal path indicated by a reference sign B.

An exemplary application of the inventive functionality can be as follows:

1) The user is in a soundscape comprising sound properties, i.e. spectral levels and modulation spectrum, allowing the inventive hearing system to recognize them as one of its acoustic cases where to check modulation sensibility of the user.

2) In a next step, the hearing system prompts the user by means of a signal which the user has learnt to be indicating that now an audible or inaudible modification to the sound is applied, for example a jingle.

3) Afterwards, during a subsequent short time period, for example 3 seconds, the user listens and checks whether he hears a modification or not.

4) Afterwards, the user notifies the hearing system via an input means if he has heard a sound modification or not.

An additional application of the exemplary functionality can be as follows: The user is introduced to different kinds of sound modifications, wherein the modifications are applied strongly, i.e. a large modulation depth at highly perceivable modulation rates, to sounds with which the user can easily perceive the modulations, for example white noise for amplitude modulation and spatial modulation, for example synthesized stationary vowels, for formant modulation.

The inventive method as well as the inventive hearing system enable users and hearing aid fitters as well as respective experts or developers to perform highly specific hearing performance analysis in real life. Advantageously, modulation detection testing can be executed for different acoustic properties of different parts or kinds of input signals. Real life testing involves a large advantage over laboratory testing, since the sounds, involving the material for discrimination testing, are the ones which the hearing impaired user lives with. Therefore, compared to the prior art, there are remarkably less validity questions to be answered. Hence, everyday life hearing performance can be checked easily in everyday's situation. The invention provides a method and a hearing system allowing increased valid real life assessments of hearing or fitting solutions. In other words, hearing performance assessment can be executed with examples of relevant sounds taken from real life or directly in real life.

1. A method for performing a hearing performance assessment, the method comprising:
receiving a sound signal from a live environment;
applying a modulation scheme to the received sound signal;
providing the modulated signal to a user of a hearing aid; and
determining whether the user of the hearing aid heard the modulation scheme at least partially based on feedback from the user.

2. The method of claim 1, wherein the modulation scheme comprises:
modulation of an amplitude of the sound signal;
modulation of center frequencies of one or more spectral peaks of the sound signal; or
modulation of a phase difference or level difference of two hearing aids.

3. The method of claim 1, wherein the method further comprises:
applying one type of modulation to different frequency ranges of the sound signal.

4. The method of claim 1, wherein the sound signal comprises different frequency ranges of the signal comprise.

5. The method of claim 1, wherein the hearing system comprises a left and a right hearing aids, and said step of applying the modulation scheme comprises:
applying the modulation scheme to the left and right hearing aids separately or to both equally or weighted.

6. (canceled)

7. The method of claim 1, further comprising:
applying the modulation scheme to a portion of the signal with stepwise varied parameters of the modulation for determining a modulation detection threshold for the user and the modulated portion of the signal.

8. The method of claim 1, further comprising:
monitoring spectral input levels and modulation spectra of the acoustic properties of the sound signal.

9. (canceled)

10. A method for performing a hearing device assessment, the method comprising:
receiving sound signal from a real life environment;
modulating the received sound signals;
providing, via a hearing aid device, the received sound signal to a user;
providing, via the hearing aid device, the modulated sound signal to the user; and
determining whether the user hears a difference between the received sound signal and the modulated sound signal based on receiving feedback from the user indicating the user heard the difference.

11. The method of claim 10, wherein modulating includes at least one of the following:
modulated of an amplitude of the sound signal;
modulation of center frequencies of the sound signal; and
modulation of a phase difference; or
any combination thereof.

12. The method of claim 10, wherein the hearing aid device is a right hearing aid device, and wherein the method further comprises:
providing, via a left hearing device, the received sound signal to the user;
providing, via a left hearing device, the modulated sound signal to a user; and
determining whether the user hears a difference between the received sound signal and the modulated sound signal based on receiving feedback from the user indicating the user heard the difference in either the left or right hearing device.
